(19) United States
(12) Patent Application Publication
(10) Pub. No.: US 2023/0069429 A1
(43) Pub. Date: Mar. 2, 2023

(54) SIZE-EXCLUSIVE DOSE-CONTROLLABLE DRUG DELIVERY IMPLANT

(71) Applicants: Yoonjee Park, Montgomery, OH (US); Xingyu He, Cincinnati, OH (US); S. Kevin Li, Cincinnati, OH (US)

(72) Inventors: Yoonjee Park, Montgomery, OH (US); Xingyu He, Cincinnati, OH (US); S. Kevin Li, Cincinnati, OH (US)

(21) Appl. No.: 17/985,828

(22) Filed: Nov. 12, 2022

Related U.S. Application Data
(62) Division of application No. 16/396,048, filed on Apr. 26, 2019.
(60) Provisional application No. 62/664,965, filed on May 1, 2018.

(51) Int. Cl. A61K 41/00; A61N 5/06; A61K 9/127; A61K 9/00; A61K 9/06; A61K 9/51; B29D 23/00

(52) U.S. Cl. CPC A61K 41/0028; A61N 5/062; A61K 9/127; A61K 9/0092; A61K 9/06; A61K 9/5153; B29D 23/00; A61K 9/51; B29K 2105/0061

(57) ABSTRACT

An implant device includes a polymer tube including an enclosed inner space, and a mixture of a hydrogel and a plurality of nanoparticles within the enclosed inner space. Each of the plurality of nanoparticles includes a shell, payload within the shell, and one or more photothermal agents on a surface of the shell. A wall of the polymer tube includes one or more layers of nanoporous polymer sheets including a plurality of pores. The dimension of the nanoparticles is greater than the dimension of the pores, and the dimension of the payload is smaller than the dimension of the pores.

### CROSS-REFERENCE TO RELATED APPLICATIONS

This application is a divisional application of U.S. application Ser. No. 16/396,048, which claims priority to U.S. Provisional Application No. 62/664,965 filed on May 1, 2018, the entire contents of which are herein incorporated by reference.

### BACKGROUND

Current therapy to treat chronic or recurrent diseases involves multiple frequent injections. Not only is this method painful and inconvenient for patients but also increases complications due to initial high dosage or infections. Current efforts in the area of drug delivery include pulsatile release formulations in which the drug is released “on-demand” over a long period of time (>6 months) in a controlled manner only when therapeutic intervention is needed. This strategy can save drug to be effective for a long time but also control the dosage as opposed to continuous (or sustained) release. External triggering mechanisms such as light or ultrasound have been combined with drug cargo, usually nano/micron particles to deliver drug. However, challenges still remain in terms of stability of the complexes inside the body, biocompatibility, safety, and therapeutic efficacy at the target lesion.

### SUMMARY OF THE INVENTION

The present disclosure is directed to biodegradable polymeric implant which contains light-activatable liposomal drug.

The biodegradable polymeric implant comprises mainly three components: i) light activatable particles (LAP), ii) hydrogel for LAP dispersant, and iii) polymer tube.

The light-activatable particle is composed of a liposome with phospholipid shell, drug that is encapsulated in the core of the liposome, and gold nanoparticles on the surface of the liposome. When the surface of the particle is irradiated by near-infrared laser, the gold nanoparticle generates enormous heat, i.e. plasmonic photothermal effect, and reversibly melts the lipid shell structure. As a result, drug inside elutes outside the shell. Although we have used gold nanoparticles as photothermal agents in our experiments, other materials can be used such as photothermal dyes, i.e. indo-cyanine green.

The light activatable particles (LAP) can be suspended in aqueous solutions or hydrogels. Hydrogel may provide better structural integrity and stability against passive leakage.

The inventors also created a biodegradable polymer tube to store the LAPs. The implant prevents the LAPs from being cleared by body fluid. The implant encapsulation also provides a defined structure (location) for laser activation, which will be practical for clinical applications. The tube is made out of thin film of biodegradable polymer, i.e. poly lactic-glycolic acid, poly lactic glycolic acid, the combination of both, or poly caprolactone, etc. The thin film is rolled using a cylindrical template to create a hollow tube.

The LAPs dispersed in a saline buffer solution are injected into the tube and both ends are closed. The LAP dispersion also can be polymerized to become a hydrogel to provide a structure and control the drug release kinetics.

### BRIEF DESCRIPTION OF THE DRAWING

The components in the drawings are not necessarily to scale relative to each other. Like reference numerals designate corresponding parts throughout the several views.

FIG. 1 describes schematic of a micro-implant injected into an eye, according to one or more embodiments shown and described herein;

FIG. 2A depicts a cross-sectional view of a micro-implant, according to one or more embodiments shown and described herein;

FIG. 2B depicts structure of an LAP activated by the light, according to one or more embodiments shown and described herein;

FIG. 2C is a confocal fluorescence microscope image of the LAPs, showing the lipid shell, and payload (drug or dye), according to one or more embodiments shown and described herein;

FIG. 2D is an enhanced dark-field image, showing the existence of gold nanoparticles 212 on the LAPs, according to one or more embodiments shown and described herein;

FIG. 2E depicts LAPs embedded in hydrogel and drug released by laser, according to one or more embodiments shown and described herein;

FIG. 3 demonstrates the production process of the biodegradable polymer tube and the final product fit in a syringe needle, according to one or more embodiments shown and described herein;

FIG. 4A is a scanning electron microscopy image, according to one or more embodiments shown and described herein;

FIG. 4B is a scanning electron microscopy image, according to one or more embodiments shown and described herein;

FIG. 4C is a scanning electron microscopy image, according to one or more embodiments shown and described herein;

FIG. 4D is a scanning electron microscopy image, according to one or more embodiments shown and described herein;

FIG. 5A demonstrate size-exclusiveness of the micro-implant created using 0.1 PEG/PLGA polymer tube of FIG. 4C.

FIGS. 5A and 5B demonstrate size-exclusiveness of the micro-implant created using 0.1 PEG/PLGA polymer tube of FIG. 4C.

FIG. 6 depicts drug release kinetics when the micro-implant is irradiated by a laser; and

FIG. 7 shows stability of the micro-implant against passive leakage in the vitreous of live rabbit eyes.

### DETAILED DESCRIPTION

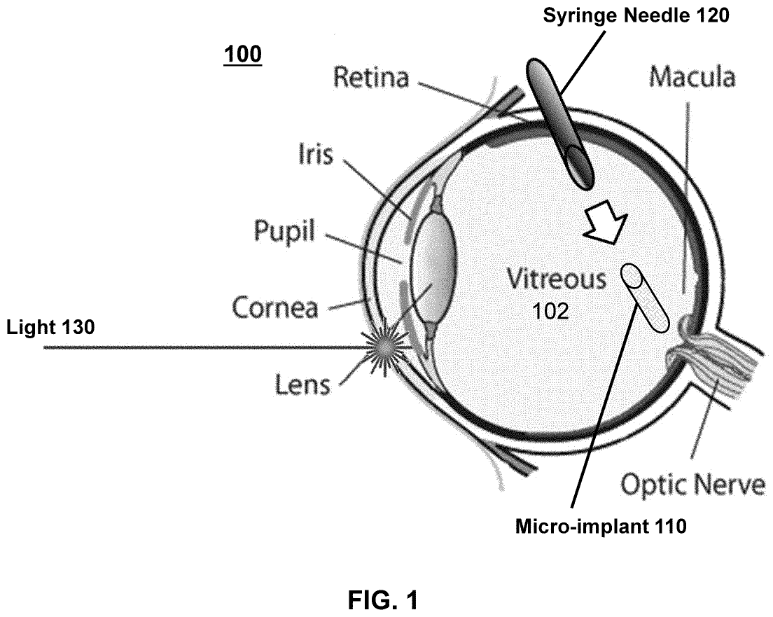

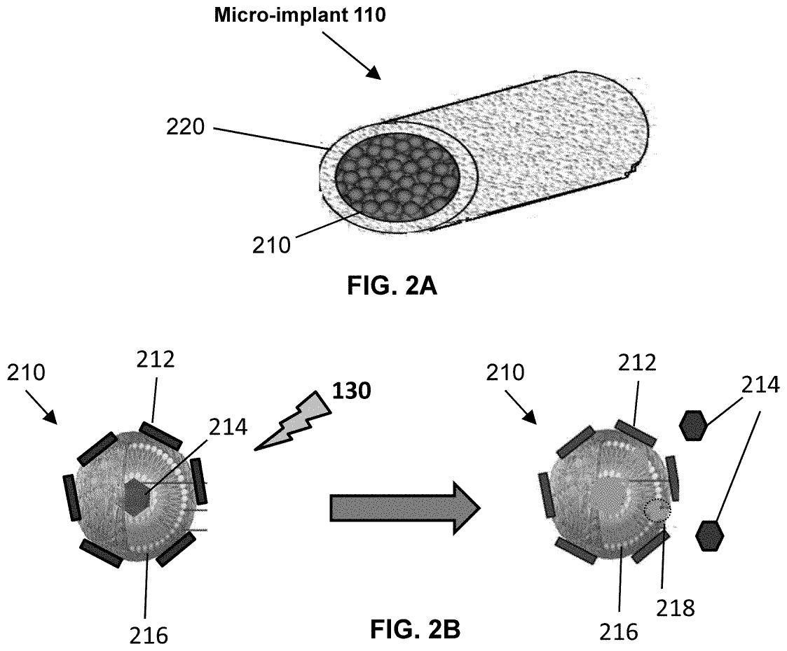

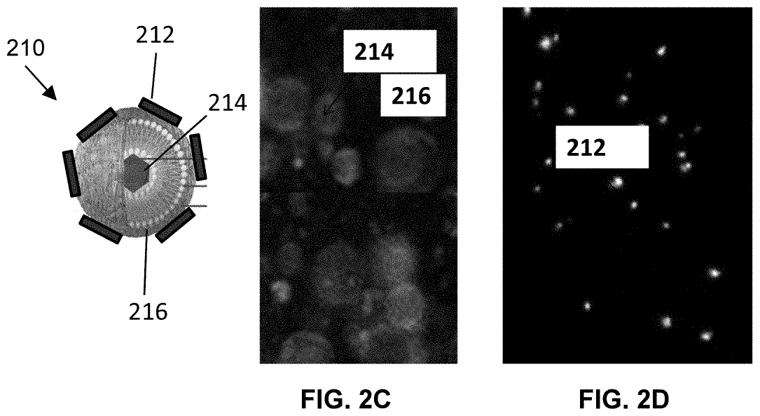

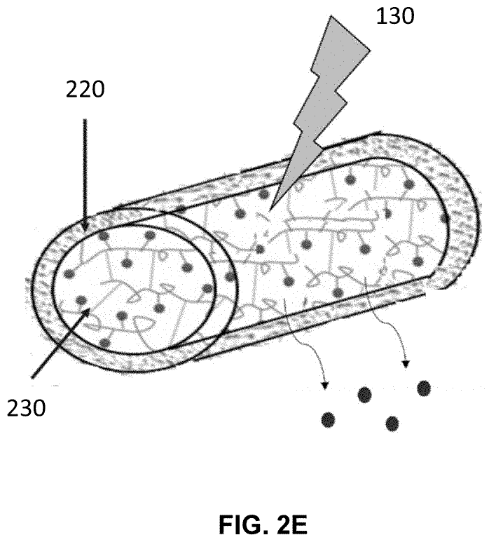

FIG. 1 describes schematic of a micro-implant 110 injected into an eye 100, according to one or more embodiments shown and described herein. As shown in FIG. 1, the micro-implant 110 may be injected into a vitreous 102 of the eye 100 by a syringe needle 120. The micro-implant 110 is fitted into the syringe needle 120, which allows one to inject into the body without incision surgery with a knife. For example, the micro-implant 110 may be fitted into the syringe needle 120 with a size of 18 G or smaller. The micro-implant 110 may include a plurality of LAPs 210, or liposomes as shown in FIGS. 2A and 2B, each of which contains payload, such as drug therein. Details of the LAPs will be described in detail with reference to FIGS. 2A and 2B below. After the micro-implant 110 is injected into the vitreous 102, a light 130 (e.g., a laser) may be projected at the eye 100. The plurality of LAPs 210 in the micro-implant 110 are light-activatable, and thus, release drug outside of the LAPs 210 in response to the projection of the laser 130. Accordingly, an amount of drug release may be controlled by an external light. The drug released through the micro-implant 110 is delivered to the target of interest to treat eye diseases, e.g., age-related macular degeneration, retinopathy, retinoblastoma, etc. The implant may be injected in other tissues or organs for different applications

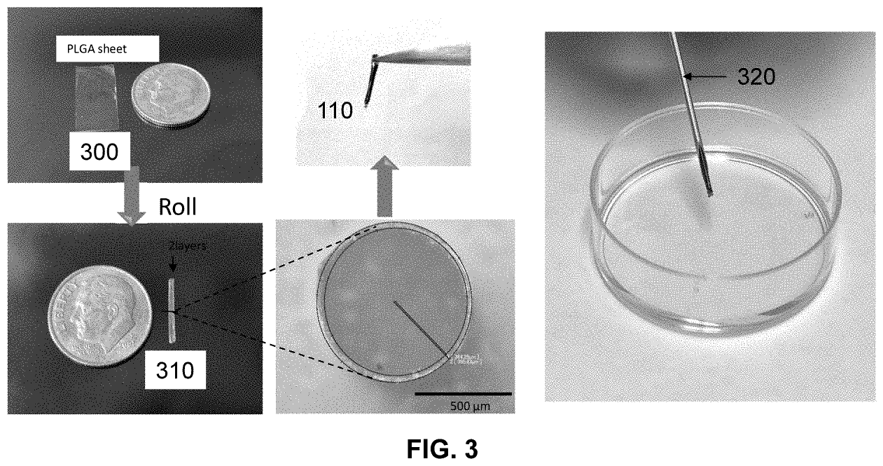

FIG. 2A depicts a cross-sectional view of the micro-implant 110. The micro-implant 110 includes a polymer tube 220. The polymer tube 220 is an enclosed structure, and thus has an enclosed inner space. The polymer tube 220 provides structure for the micro-implant 110 as well as prevents passive leakage of materials (e.g., LAPs 210) contained within the polymer tube 220. A mixture of the plurality of LAPs 210 and a hydrogel are placed within the enclosed inner space. The polymer tube 220 may be made of a polymer sheet having nanoscale pores. The method for manufacturing the polymer tube 220 will be described in detail with reference to FIG. 3 below.

FIG. 2B depicts structure of the LAP 210 activated by the light 130, according to one or more embodiments shown and described herein. The LAP 210 includes a shell 216, one or more photothermal agents 212 on the surface of the shell 216, and payload within the shell 216.

FIG. 2C is a confocal fluorescence microscope image of the LAPs, showing the lipid shell 216, and payload (drug or dye) 214, with green and red color, respectively. For the shell 216, phospholipids, fatty acids, polymeric lipids, and cholesterol have been used. The lipids used for this invention is distearoyl phosphatidylcholine (DSPC), cholesterol, stearylamine, and distearoyl glycero phosphoethanolamine-polyethylene glycol 5000 (DSPE-PEG 5K). Stearylamine is used for providing positive charge. Positive charge is utilized to adsorb negatively charged gold nanoparticles electrostatically. DSPE-PEG5K is used to provide steric stability again aggregation. The lipids may be of natural and/or synthetic origin. Such lipids include, but are not limited to, fatty acids, lysolipids, dipalmitoylphosphatidylcholine, phosphatidylcholine, phosphatidic acid, sphingomyelin, cholesterol, cholesterol hemisuccinate, tocopherol hemisuccinate, phosphatidylethanolamine, phosphatidyl-inositol, lysolipids, sphingomyelin, glycosphingolipids, glucolipids, glycolipids, sulphatides, lipids with ether and ester-linked fatty acids, diacetyl phosphate, stearylamine, distearoylphosphatidylcholine, phosphatidylserine, sphingomyelin, and cardiolipin. For the cationic (positively-charged) lipids, diacyl ethylphosphatidylcholine with chain fatty acids of 12-18 carbons in length, dimethyldioctadecylammonium, dimethylaminoethane-carbamoyl cholesterol hydrochloride, and di-O-octadecenyl-3-trimethylammonium propane can be used. The mole percentage of the cationic lipid can vary from 1% to 50%.

The liposomes can be synthesized via various methods, including sonication, stirring, extrusion, or reverse-phase method. In this invention, reverse-phase method is mainly used because this tends to create micron-sized big liposomes, which are beneficial against passive leakage through the implant pore structure.

FIG. 2D is an enhanced dark-field image, showing the existence of gold nanoparticles 212 on the LAPs. Gold nanoparticles 212 were adsorbed on the shell after creation of the liposomes via electrostatic binding. Gold nanoparticles have been investigated for use of biomedical applications because of its ease of synthesis and surface modification, and biocompatibility. In addition, gold nanoparticles generate heat when exposed to incident laser illumination at wavelengths close to the surface plasmon resonance which efficiently couple the optical energy. The heat induced by laser is localized near the nanoparticles. Depending on the size and shape of nanoparticles, intensity or illumination time of laser, temperature rise can be tuned. Surface plasmon absorbance wavelength also depends on the nanoparticle's size and shape; thus, using right wavelength of laser is important to generate heat effectively. Gold nanoparticles with sphere shape have surface plasmon absorbance around 525 nm. Gold nanorods have a band at longer wavelengths around 700-1000 nm due to the plasmon oscillation of electrons along the long axis of the nanorods, in addition to the surface plasmon band around 525 nm seen in gold nanospheres. Depending on aspect ratio and size, the second band peak appears in the range of 700 nm to 1000 nm. In this disclosure, gold nanorods are used because they resonate with near-infrared light region (700 to 1000 nm), which can penetrate tissue and potentially be used clinically. Hollow gold nanoshells also can be used in the similar wavelength region.

Other possible agents are small molecular dyes, i.e. indo-cyanine green. We have preliminary results on testing the dye to trigger drug release using a near infrared laser. This dye is approved by FDA for diagnostic purposes. Squaraine and croconine dye derivatives are also known to be photothermal at the near-infrared region.

FIG. 2E depicts LAPs 210 are embedded in hydrogel 230 and drug is released by laser 130. The LAP 210 dispersion may undergo gelation via polymerization. The hydrogel 230 provides structure and stability against passive leakage compared to liposome aqueous dispersion. If the polymer tube 220 is damaged or degraded, aqueous (liquid) form will elute, which fails controlled release. On the other hand, one does not have to solely rely on the polymer tube 220 to control drug release if liposomes embedded in a hydrogel are used. The liposome dispersion is mixed with monomers and cross-linking agents, then transferred to the polymer tube 220 quickly before it polymerizes. The liposome dispersion becomes hydrogel inside the polymer tube 220 about 10 minutes. The monomer and the cross-linking agents used in this invention is acrylamide and TEMED (Tetramethylethylenediamine), respectively. The hydrogel may be collagen-based, gelatin-based, hyaluronic acid-based, pullulan-based, polyethylene glycol-based, poly lactic acid, poly glycolic acid, poly lactic glycolic acid-based, poly caprolactone-based, chitosan-based, poly ethylene oxide-based, etc.

FIG. 3 demonstrates the production process of the biodegradable polymer tube 220 and the final product 110 fit in a syringe needle 320. Such polymer include, but are not limited to, poly lactic glycolic acid (PLGA), poly lactic acid, poly glycolic acid, chitosan, cellulose, poly caprolactone, etc. These materials degrade in the body without toxicity. The choice of the materials depends on the degradation rate or other physical/chemical properties, such as resistance or light transparency. In this invention, PLGA is used. Within PLGA, high molecular weight with high L/G ratio is used to prolong the degradation period up to 12 months.

The polymer tube 220 is created by rolling a polymer thin sheet 300. How the sheet is created will be described below. The sheet is cut in a desired size and rolled on a cylindrical template to create a polymer tube (FIG. 3). When the tube lumen is filled with LAPs 210, both ends are closed using an iron or a hair straightner. The micro-implant is fit into a syringe needle 320, which allows one to inject into the body without incision surgery with a knife.

The polymer tube 220 has nanoporous structure to exclusively release drug 214, not the LAPs 210. The nanopores are bigger than the drug 214 but smaller than the LAP 210 in size. The nanoporous polymer sheets were synthesized by solvent casting particulate leaching method.

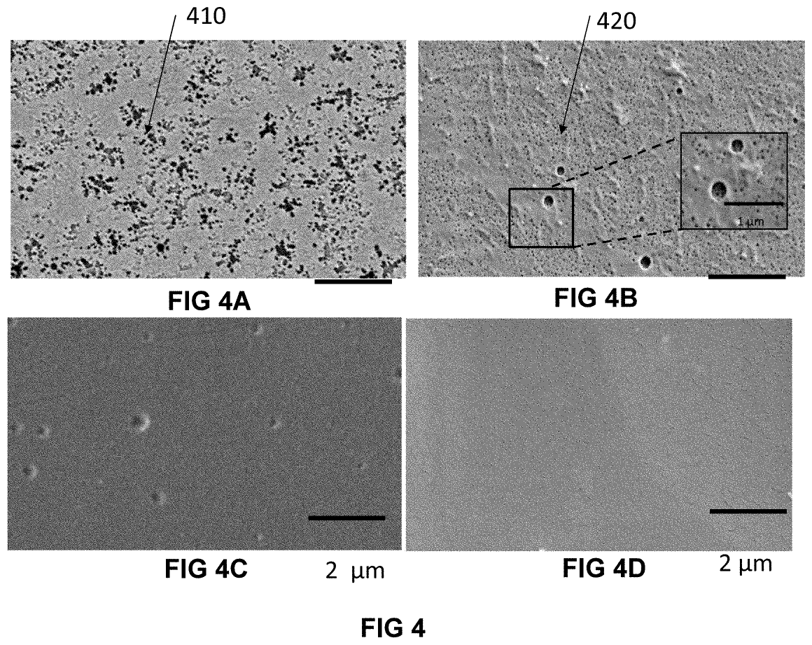

FIGS. 4A and 4B are scanning electron microscopy images, showing the uniform nanoporous structure 410 and 420, respectively. The nanopores are created by using a porogen (pore generation agent). In this invention, polyethylene glycol (PEG) is used as a porogen. Depending on the ratios of the PEG to PLGA, the pore size can be controlled. FIGS. 4A to 4D demonstrates that the pore size decreases from −100 nm to 50 nm to less than 10 nm, and none when the ratios are 0.2, 0.17, 0.1, and 0 by weight.

The mixture of PLGA and PEG solution in an organic solvent is transferred into a mold. The mixture in a mold floats at the water surface in a bath sonicator and is sonicated at low temperature. The top of the mold is covered by parafilm to avoid water droplets during bath sonication. The sonicated mixture is air dried overnight in the fume hood to evaporate the organic solvent. The dry sheet is peeled off and is soaked in deionized (DI) water with stirring overnight to dissolve PEG in water. The PLGA nanoporous sheet is finally air dried at room temperature overnight.

Dependent on the pore size of the polymer tube, one can selectively release different sizes of payload. The pore size can be controlled by using different solvents or porogens (pore generation agents). For example, if the pore size of the polymer sheet is 50 nm, drug molecules (1-15 nm) will pass through the tube while liposomes (>100 nm) will not.

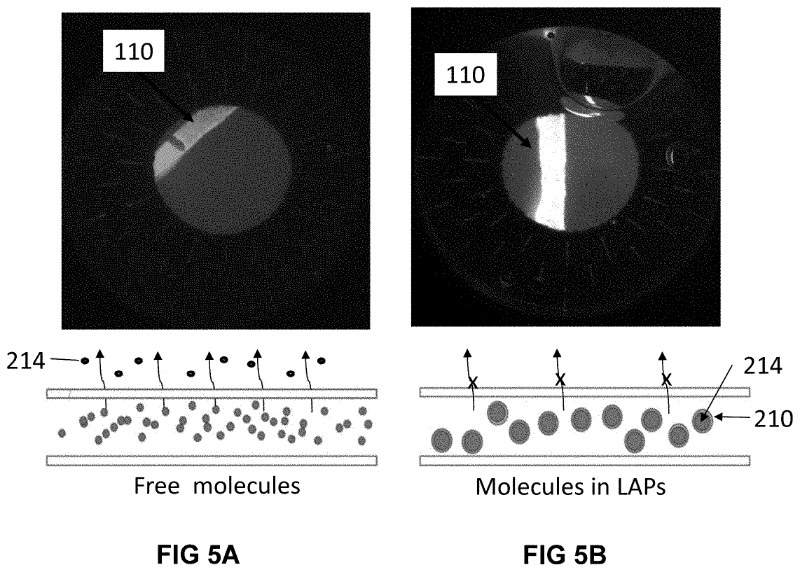

FIGS. 5A and 5B demonstrate size-exclusiveness of the micro-implant created using 0.1 PEG/PLGA polymer tube (FIG. 4C). When free dye molecules are filled in the polymer tube, within 24 hours, a significant amount of the dye molecules are detected outside the implant. On the other hand, when the dye molecules are inside liposomes, the dye molecules are not detected outside the implant. The results indicate the pore size of the polymer tube is less than the liposome but bigger than the molecule.

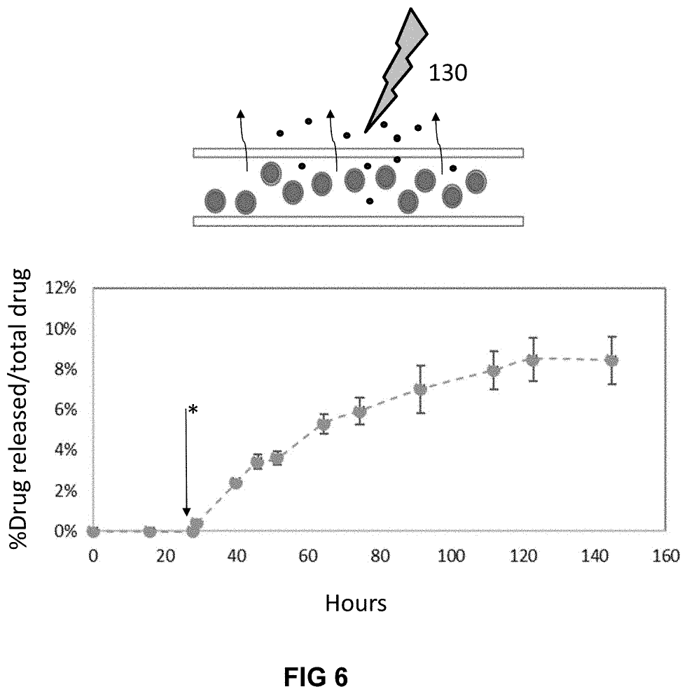

FIG. 6 includes drug release kinetics when the micro-implant 110 is irradiated by a laser 130. The % release is controlled by the irradiation time and power. In order to induce drug (payload) release, local heating by laser illumination of gold nanoparticles on the surface is utilized. Gold nanoparticles exposed to incident laser illumination at wavelengths close to the surface plasmon resonance efficiently couple the optical energy and generate heat. For the wavelength of the laser, near-infrared light region (700 nm-1000 nm) is used for clinical purposes and correspondingly gold nanorods are used to match the plasmon band. The heat induced by laser is localized near the nanoparticles. The temperature rise upon laser illumination depends on power, duration, or whether the illumination is continuous wave or pulsed. The heat changes nanoparticle membrane (shell) structure by “melting” to become more fluidized. The payload inside then releases. Lipid shell has a chain-melting temperature over which the membrane structure becomes fluidized, depending on the lipid chain length.

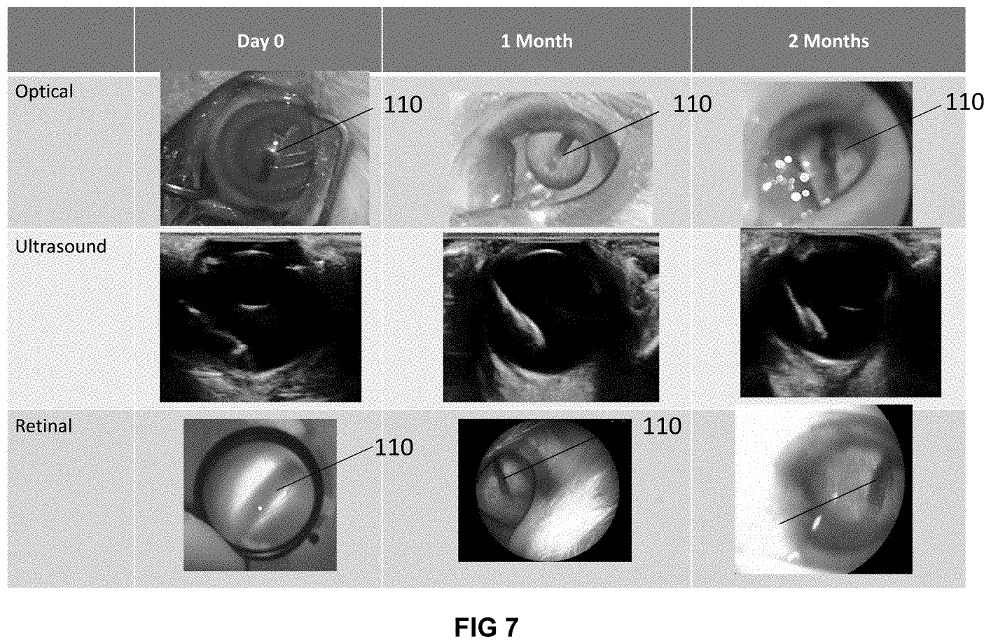

FIG. 7 shows stability of the micro-implant 110 against passive leakage in the vitreous 102 of live rabbit eyes. No significant dye is leaked across the implant at least for two months. The location of the implant does not change for two months, confirmed by ultrasound.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

What is claimed is:

1. A method for releasing payload in an implant device, the method comprising:
placing the implant device in a syringe needle;
injecting the implant device into an object; and
irradiating a laser at the implant device,
wherein the implant device comprise:
a polymer tube including an inner space; and
a plurality of particles within the inner space, wherein each of the plurality of particles includes:
a lipid shell;
drug within the shell; and
one or more photothermal agents.

2. The method of claim 1, wherein:
a wall of the polymer capsule includes one or more layers of porous polymer sheets;
the porous polymer sheet includes a plurality of pores;
a dimension of the particles in any direction is greater than a dimension of the pores; and
a dimension of the payload is smaller than a dimension of the pores.

3. The method of claim 1, wherein:
each of the plurality of particles is configured to output the drug outside the lipid shell in response to the irradiated laser such that the drug go through the plurality of pores.

4. The method of claim 1, wherein the laser has a wavelength between 700 nanometers and 1,000 nanometers.

5. The method of claim 1, wherein the phototermal agent is a gold nanorod.

6. The method of claim 1, wherein the polymer capsule is biodegradable.

7. The method of claim 1, wherein the polymer capsule includes at least one of poly lactic-glycolic acid, or poly caprolacton.
